(19) United States
(12) Patent Application Publication
(10) Pub. No.: US 2026/0295522 A1
(43) Pub. Date: Oct. 1, 2026

(54) ETHYLENE OXIDE ABATEMENT SYSTEMS

(71) Applicant: Boston Scientific Scimed, Inc., Maple Grove, MN (US)

(72) Inventors: Lauren Sfakis Lydecker, Upton, MA (US); Joseph Thomas Delaney, JR., Minneapolis, MN (US); Rick William Dorn, Plymouth, MN (US); Liming Dong Crandall, Lino Lakes, MN (US); Vishal Jayesh Bavishi, Natick, MA (US)

(73) Assignee: Boston Scientific Scimed, Inc., Maple Grove, MN (US)

(21) Appl. No.: 19/632,953

(22) Filed: Mar. 30, 2026

Related U.S. Application Data
(60) Provisional application No. 63/780,856, filed on Mar. 31, 2025.
(60) Provisional application No. 63/850,298, filed on Jul. 24, 2025.

(51) Int. Cl. B01D 53/86; A61L 2/206; A61L 103/15; B01J 31/10; B65D 81/26

(52) U.S. Cl. CPC B01D 53/8668; A61L 2/206; B01J 31/10; B65D 81/267; A61L 2103/15; A61L 2202/13; A61L 2202/181; B01D 2251/70; B01D 2255/70; B01D 2257/708; B01D 2259/4533; B01J 2531/002

(57) ABSTRACT

A system for abatement of sterilizing gas may include a device package having a device disposed therein and a sterilizing gas abatement member disposed adjacent to the device package. The sterilizing gas abatement member may comprise a base material and a catalyst comprising sulfonic acid functional groups. The catalyst is configured to abate a sterilizing gas.

### CROSS-REFERENCE TO RELATED APPLICATIONS

This application claims the benefit of priority under 35 U.S.C. § 119 of U.S. Provisional Application No. 63/850,298, filed Jul. 24, 2025, and U.S. Provisional Application No. 63/780,856, filed Mar. 31, 2025, the entire disclosure of which is hereby incorporated by reference.

### TECHNICAL FIELD

The present disclosure pertains to medical devices, and methods for manufacturing and packaging medical devices. More particularly, the present disclosure pertains to ethylene oxide abatement systems.

### BACKGROUND

A wide variety of medical devices have been developed for medical use, and more specifically, for intravascular use. Some of these devices include guidewires, catheters, and the like. Most of these devices are required to be sterilized prior to use. Depending on the device type, material of construction, and device design, these devices are sterilized by any one of a variety of different sterilization methods, including, but not limited to, ethylene oxide (EtO) sterilization. EtO sterilization is a widely used sterilization method, among others, for medical device sterilization. There is an ongoing need to improve abatement technologies to reduce ethylene oxide exposure post-sterilization.

### BRIEF SUMMARY

This disclosure provides design, material, manufacturing, and packaging methods for ethylene oxide abatement. A sterilization abatement member may be positioned along with, near, or in proximity to the medical device primary package post-ethylene oxide sterilization.

In an example, a system for abatement of sterilizing gas may comprise a device package having a device disposed therein and a sterilizing gas abatement member disposed adjacent to the device package, where the sterilizing gas abatement member may comprise a base material and a catalyst comprising sulfonic acid functional groups, and the catalyst may be configured to abate a sterilizing gas present adjacent to the device package.

Alternatively, or additionally to any of the examples above, in another example, the base material may comprise at least one of chitosan, cellulose, a cellulose derivative, other polysaccharides, or glycosaminoglycans.

Alternatively, or additionally to any of the examples above, in another example, the catalyst may comprise polystyrene sulfonic acid (PSSA).

Alternatively, or additionally to any of the examples above, in another example, the sterilizing gas abatement member may have a porosity in a range of about 10-90%.

Alternatively, or additionally to any of the examples above, in another example, the sterilizing gas abatement member may have a porosity in a range of about 40-80%.

Alternatively, or additionally to any of the examples above, in another example, the sterilizing gas may comprise ethylene oxide

Alternatively, or additionally to any of the examples above, in another example, the sterilizing gas abatement member may be configured as a blanket.

Alternatively, or additionally to any of the examples above, in another example, the sterilizing gas abatement member may be configured as a liner.

Alternatively, or additionally to any of the examples above, in another example, the device package may be disposed within the sterilizing gas abatement member.

Alternatively, or additionally to any of the examples above, in another example, the sterilizing gas abatement member may be disposed about the device package.

Alternatively, or additionally to any of the examples above, in another example, the device package may comprise a medical device package having a medical device therein.

In an example, a method of manufacturing a sterilizing gas abatement member may comprise mixing a base material with an organic solvent and water solution to form a first mixture, adding a catalyst comprising mineral acid functional groups to the first mixture to form a second mixture, pouring the second mixture into a mold, and removing water from the second mixture to form a porous matrix.

Alternatively, or additionally to any of the examples above, in another example, removing water may comprise freeze-drying the second mixture.

Alternatively, or additionally to any of the examples above, in another example, removing water may comprise at least one of spray drying, oven drying, vacuum drying, fiber spinning, or using supercritical CO2.

Alternatively, or additionally to any of the examples above, in another example, the catalyst may be added in a molar ratio of one-to-one or less to the base material.

Alternatively, or additionally to any of the examples above, in another example, the acid may comprise acetic acid, lactic acid, or hydrochloric acid.

In an example, a system for abatement of sterilizing gas may comprise a device package having a device disposed therein and a sterilizing gas abatement member disposed adjacent to the device package, where the sterilizing gas abatement member may comprise a base material and a catalyst comprising sulfonic acid functional groups, and the catalyst may be configured to abate a sterilizing gas present adjacent to the device package.

Alternatively, or additionally to any of the examples above, in another example, the base material may comprise at least one of chitosan, cellulose, or a cellulose derivative, other polysaccharides, glycosaminoglycans, or other related systems.

Alternatively, or additionally to any of the examples above, in another example, the catalyst may comprise polystyrene sulfonic acid (PSSA).

Alternatively, or additionally to any of the examples above, in another example, the sterilizing gas abatement member may have a porosity in a range of about 10-90%.

Alternatively, or additionally to any of the examples above, in another example, the sterilizing gas abatement member may have a porosity in a range of about 40-80%.

Alternatively, or additionally to any of the examples above, in another example, the sterilizing gas

Alternatively, or additionally to any of the examples above, in another example, the sterilizing gas abatement member may be configured as a liner.

Alternatively, or additionally to any of the examples above, in another example, the sterilizing gas may comprise ethylene oxide.

Alternatively, or additionally to any of the examples above, in another example, the device package may be disposed within the sterilizing gas abatement member.

Alternatively, or additionally to any of the examples above, in another example, the sterilizing gas abatement member may be disposed about the device package.

In an example, a sterilizing gas abatement system may comprise a container configured to hold one or more sterilized devices and a sterilizing gas abatement member positioned within or surrounding the container, where the sterilizing gas abatement member may comprise a porous matrix formed from a base material and a catalyst, the base material may provide structural integrity to the porous matrix, the catalyst may comprise sulfonic acid functional groups, and the catalyst may be configured to convert ethylene oxide gas to ethylene glycol.

Alternatively, or additionally to any of the examples above, in another example, the porous matrix may have a porosity in the range of about 10-90%.

Alternatively, or additionally to any of the examples above, in another example, the base material may comprise at least one of chitosan, cellulose, or a cellulose derivative, other polysaccharides, glycosaminoglycans, or other related systems.

Alternatively, or additionally to any of the examples above, in another example, the catalyst may comprise polystyrene sulfonic acid (PSSA).

Alternatively, or additionally to any of the examples above, in another example, the catalyst may be present in a molar ratio of one-to-one or less to the base material.

In an example, a method of manufacturing a sterilizing gas abatement member may comprise mixing a base material with an acid and water solution to form a first mixture, adding a catalyst comprising sulfonic acid functional groups to the first mixture to form a second mixture, pouring the second mixture into a mold, and removing water from the second mixture to form a porous matrix.

Alternatively, or additionally to any of the examples above, in another example, removing water may comprise freeze-drying the second mixture.

Alternatively, or additionally to any of the examples above, in another example, removing water may comprise at least one of spray drying, oven drying, vacuum drying, fiber spinning, or using supercritical CO2.

Alternatively, or additionally to any of the examples above, in another example, the catalyst may be added in a molar ratio of one-to-one or less to the base material.

Alternatively, or additionally to any of the examples above, in another example, the acid may comprise acetic acid, lactic acid, or hydrochloric acid.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically depicts an example medical device disposed within a medical device package post-sterilization;

FIG. 2 is an example chemical reaction by which the sulfonic acid functional groups of a catalyst catalyze the conversion of ethylene oxide to ethylene glycol;

FIG. 3 schematically depicts a plurality of medical device packages with a sterilizing gas abatement member disposed adjacent to the medical device packages;

FIG. 4 schematically depicts a plurality of medical device packages with a sterilizing gas abatement member disposed adjacent to the medical device packages;

FIG. 5 schematically depicts a plurality of medical device packages with a sterilizing gas abatement member disposed adjacent to the medical device packages;

FIG. 6A is a graphical representation of experimental data providing relative ethylene oxide concentration decrease over a period of time;

FIG. 6B is an enlarged region of the graph of FIG. 6A taken at detail B of FIG. 6A; and

FIG. 7 is a graphical representation of experimental data providing relative ethylene oxide concentrations of the ambient atmosphere and inside a cardboard box wrapped with ethylene oxide abatement members over a period of time.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Medical devices such as guidewires, catheters, balloon catheters, stents, stent delivery systems, and the like are sterilized prior to use to eliminate the presence of microorganisms and to provide efficacious and safe use. A number of different sterilization techniques may be used. One example sterilization technique is the use of ethylene oxide (EtO) gas. Ethylene oxide may be highly effective for most materials that are sensitive to humidity and temperature. Ethylene oxide is also strictly regulated by the Occupational Safety and Health Administration (OSHA) and Environmental Protection Agency (EPA) due to its hazardous properties, including, but not limited to, being flammable and carcinogenic. While effective for sterilization, residual ethylene oxide gas can be present (e.g., off-gas) from the devices and/or packaging after the sterilization procedure. In general, the relative amount of residual ethylene oxide gas present may be considered low. However, in a manufacturing setting where relatively large quantities of sterilized and packaged medical devices may be present, the levels of residual ethylene oxide gas can rise to undesirable levels.

A common abatement technology for ethylene oxide is chemical gas scrubbers that use sulfuric acid as a catalyst which stabilizes and breaks down ethylene oxide into ethylene glycol and ethylene glycol derivatives. The EPA requires additional personal protective equipment (PPE), such as a self-contained breathing apparatus or a supplied airline respirator, to mitigate potential inhalation exposure risks to workers involved in the ethylene oxide sterilization process. Respirators are required for exceedances of occupational exposure limit of 1 ppm (1,000 ppb) (8-hour time weighted average) and short-term exposure limit (“STEL”) of 5 ppm (5,000 ppb) (15-minute time weighted average) in commercial sterilization facilities and healthcare facilities. Per the new FIFRA Interim decision, the occupational exposure limit will be lowered incrementally over the next ten years from 1 ppm (1000 ppb) to 0.1 ppm (100 ppb). It also requires real-time monitoring of ethylene oxide with a limit of quantification at or below 0.1 part per million (100 parts per billion (ppb)). The workplace action level is proposed at 100 ppb in the workplace, above which the workers are required to wear PPE. For worker safety and to comply with EPA regulations, the sterilized goods are often stored and allowed to off-gas (emit residual ethylene oxide). Disclosed herein are systems for abatement of residual sterilizing gas, for example, ethylene oxide. While the systems and methods are described with respect to sterilized medical devices, it is contemplated that the systems and methods may be used for ethylene oxide abatement in other applications where ethylene oxide is present.

Medical devices may be packaged in a number of different ways. In some cases, medical devices may be packaged in a primary package prior to sterilization. Primary packaging may include, but is not limited to, pouches, blister packs, bags, or the like. After sterilization, the primary packages may be grouped and placed into secondary packaging. Secondary packaging may include, but is not limited to, cardboard boxes, plastic trays, or the like. Each secondary packaging container may include one or more medical devices each disposed within a primary package. Further, the secondary packaging containers may be grouped and placed into tertiary packaging. Tertiary packaging containers may include, but are not limited to, pallets, stretch wrap, or other containers. Each tertiary packaging container may include two or more secondary packaging containers.

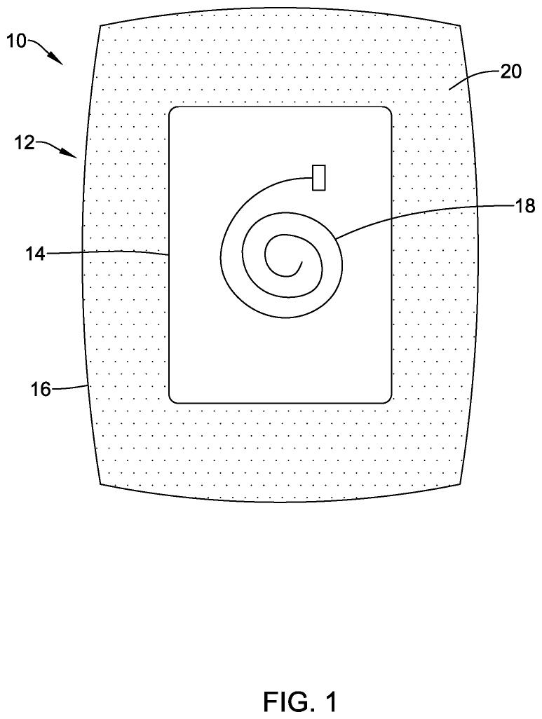

FIG. 1 schematically depicts an example system 10. The system 10 may include a medical device package 12. In this example, the medical device package 12 may include an inner packaging member or pouch 14 and an outer packaging member or pouch 16. Examples are contemplated that utilize more or fewer components. For example, the outer packaging member 16 may be omitted. A medical device 18, depicted schematically, may be disposed within the medical device package 12. The medical device 18 may be any of a variety of different medical devices such as guidewires, catheters, balloon catheters, stents, stent delivery systems, and the like. These are just examples. Other forms of medical devices are contemplated. In this example, the medical device 18 is shown disposed within the inner packaging member/pouch 14. In some examples, the outer packaging member/pouch 16 may be formed from a sterilizing gas abatement substance or member 20. In such an example, the inner packaging member/pouch 14 may be placed in the outer packaging member/pouch 16 after a sterilization procedure. However, the outer packaging member/pouch 16 need not be formed from a sterilizing gas abatement substance or member 20.

The sterilizing gas abatement member 20 may have a variety of forms. In general, the sterilizing gas abatement member 20 may be configured to react with ethylene oxide gas and neutralize the ethylene oxide gas. In some examples, the sterilizing gas abatement member 20 may react with ethylene oxide gas to form ethylene glycol and/or such derivates. However, other chemical reactions may occur. In some instances, the sterilizing gas abatement member 20 may include a porous material such as, but not limited to, a foam and a catalyst.

Generally, the sterilizing gas abatement substance 20 may include a base material or bulking agent. The base material may provide structural integrity to the sterilizing gas abatement member 20 to allow the sterilizing gas abatement member 20 to be free-standing. The base material may form a matrix or framework of the sterilizing gas abatement member 20. The sterilizing gas abatement member 20 may have a porous structure that allows ethylene oxide gas to freely diffuse therethrough. Illustrative base materials may include, but are not limited to, chitosan, cellulose, cellulose derivatives, other polysaccharides, glycosaminoglycans (e.g., hyaluronic acid or heparin), alginates, or the like. In some cases, the base material may be biodegradable. The base material may have a porosity (e.g., percentage of void space relative to its total volume) in the range of about 10-90%. However, the porosity may be less than 10% or greater than 90%, as desired.

The sterilizing gas abatement member 20 may further include a catalyst or functional component that activates or performs the desired chemical reaction. Namely, the catalyst converts the ethylene oxide to ethylene glycol and/or other derivatives. The catalyst may include mineral acid functional groups which, in some cases, may be, but are not limited to, sulfonic acid groups, sulfuric acid groups, or phosphoric acid groups. Some illustrative catalysts may include, but are not limited to, polystyrene sulfonic acid (PSSA), dyes with sulfonic acid end groups, or the like. The catalyst may be incorporated into the base material through an ionic interaction (e.g., a coacervate) between the base material and the catalyst and/or through chain entanglement. A coacervate refers to the structural formation created through ionic interactions between oppositely charged components. More particularly, it describes the structure formed when the positively charged groups of the base material (such as, but not limited to, the primary amines in chitosan) interact with negatively charged functional groups (such as, but not limited to, the sulfonate groups in PSSA). This may create a sterilizing gas abatement member 20 where the catalytic sites are distributed throughout the porous structure of the base material. As ethylene oxide diffuses through the sterilizing gas abatement member 20, the sulfonic acid functional groups catalyze the conversion of ethylene oxide to ethylene glycol.

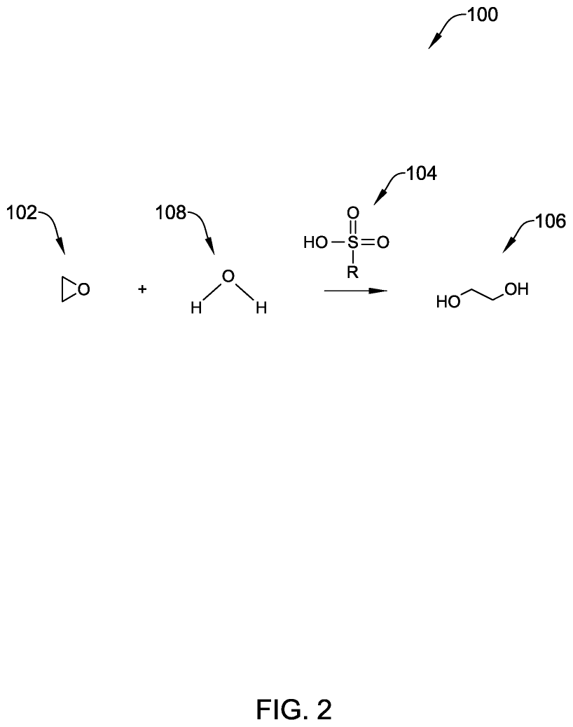

FIG. 2 illustrates an example chemical reaction 100 by which the sulfonic acid functional groups of the catalyst 104 catalyze the conversion of ethylene oxide 102 to ethylene glycol 106. The R group of the catalyst 104 represents attachment of the sulfonic acid group to the remainder of the molecule. As described herein, in some cases, the catalyst 104 may be PSSA which contains multiple sulfonic acid function groups along its polymer chain. PSSA has the following chemical structure:

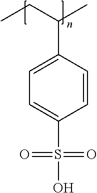

The conversion of ethylene oxide 102 to ethylene glycol 106 occurs through an acid-catalyzed ring opening reaction in which the three-membered epoxide ring of ethylene oxide 102 is cleaved by nucleophilic attack. In this reaction, a sulfonic acid group (R—SO₃H) 104 serves as the catalyst by protonating the oxygen atom of the ethylene oxide molecule 102, creating a positive charge on one of the carbon atoms of the ring. This protonation renders the carbon electrophilic and susceptible to nucleophilic attack by water 108. The sterilizing gas abatement member 20 may be hygroscopic which draws water 108 from the ambient atmosphere into the sterilizing gas abatement member 20 during the reaction. A water molecule 108 then attacks the electrophilic carbon atom, causing the ring to open. The oxygen from the water molecule 108 forms a hydroxyl group at one carbon, while the oxygen from the original epoxide forms a second hydroxyl group at the adjacent carbon, yielding ethylene glycol (HOCH₂CH₂OH) 106 as the main product with other ethylene glycol derivatives. This reaction proceeds exothermically (e.g., releases heat) and requires stoichiometric amounts of water (one equivalent of water per molecule of ethylene oxide) to complete the transformation. The reaction efficiency may be enhanced by the hygroscopic nature of the sterilizing gas abatement member 20, which may naturally attract and absorb ambient moisture from the surrounding environment, ensuring sufficient water is available for the reaction to proceed. The sulfonic acid catalyst 104 may not be consumed in the reaction and can continue to facilitate multiple conversion cycles. Thus, the sterilizing gas abatement member 20 may abate ethylene oxide 102 as long as the catalyst 104 is active.

In an example reaction, chitosan (e.g., the base material or bulking agent) is mixed with an acetic acid and water mixture. The acetic acid may have a concentration in the range of about 0.5% to about 5%. It is contemplated that other weak acids, such as, but not limited to, hydrochloric acid or lactic acid, or other organic solvents, such as, but not limited to, ethanol or methanol, may be used in place of acetic acid. Chitosan may be mixed into the acetic acid and water solution using an air mixer or other mixing device. PSSA (e.g., catalyst) may then be added to the chitosan/acetic acid/water solution to form a mixture. PSSA may be added in a one-to-one molar ratio to the chitosan. However, the catalyst may be added in various molar ratios relative to the base material, including ratios significantly less than one-to-one, such as in the range of a few percent by molar ratio to the base material. This flexibility in catalyst loading may allow for optimization of the abatement performance while maintaining the structural integrity provided by the base material. Upon addition to the chitosan/acetic acid/water solution, PSSA may form ionic cross-links with the chitosan. In some cases, a coacervate having a porous structural network may be formed. The ionic interaction forms between the positively charged primary amine groups of chitosan and the negatively charged sulfonate groups of PSSA. The resulting mixture may be a gel which is then poured into a tray or other appropriate mold to form the desired shape. For example, large molds may be used to form blankets, liners, wraps, or other structures configured to be positioned over or near a large surface area. Alternatively, molds may be used which have a smaller volume to form a plurality of smaller sterilizing gas abatement substances or members 20 that may be incorporated into a package in a manner similar to packing peanuts. These are just some examples. It is contemplated that the size and shape of the mold may be selected to suit the desired application and/or positioning of the sterilizing gas abatement member 20.

The mold may then be placed in a freezer having a temperature at or below the freezing point of water (e.g., 32° F. or 0° C.). It is contemplated that the final pore structure of the sterilizing gas abatement member 20 may be achieved by controlling the percent solids in the mixture and/or by controlling freezing mechanism (e.g., a slow ramp freeze versus a flash freeze). For example, reducing the percent solids would decrease the bulk density of the foam and resulting porous network. Increasing the percent solids would increase the bulk density of the foam and make it a more concentrated foam network. Controlling freezing of the gel can introduce different pore networks. For example, flash freezing or slow ramp freezing form small or large ice crystals on exposed surfaces of a material. Larger ice crystals potentially result in more damage to the cellular walls or porous structure of the material impacting the overall properties of the material.

Once the mixture is completely frozen, the frozen mixture may be transferred to a freeze dryer for lyophilization. Lyophilization may remove water from the mixture while maintaining the porous structure. Lyophilization is a process of creating ice structures in the freezing process which then sublime out of the gel once freeze dried. The method used to freeze the foam may create different pores that will then yield different “void” networks. The freeze-drying process may be continued and/or repeated until the material appears dry. Dryness may be confirmed when the material, the sterilizing gas abatement member 20, can be easily removed from the mold. The sterilizing gas abatement member 20 may retain some ambient moisture. Other processes may be used to dry the chitosan/acetic acid/water/catalyst mixture to yield a porous matrix, such as, but not limited to, spray drying, oven drying, vacuum drying, super critical CO₂, fiber spinning, or the like.

The resulting sterilizing gas abatement member 20 may have a highly porous structure (e.g., 70-80%) with distributed catalytic sites. Further, the chitosan (or other base material) may provide structural integrity to the sterilizing gas abatement member 20 to allow the sterilizing gas abatement member 20 to be free-standing. The sterilizing gas abatement member 20 may be shaped into various form factors, such as, but not limited to, blankets, panels, inserts, liners, wraps, boxes, or the like.

In other illustrative examples, the catalyst 104 may be dyes containing sulfonic acid functional groups. An illustrative catalyst may be Brilliant Blue, also known as erioglaucine disodium salt, having a chemical abstracts service (CAS) registry number of 3844-45-9 and the following chemical structure:

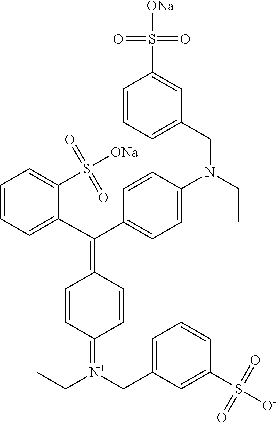

Another illustrative catalyst may be Fast Green FCF, having a CAS registry number of 2353-45-9 and the following chemical structure:

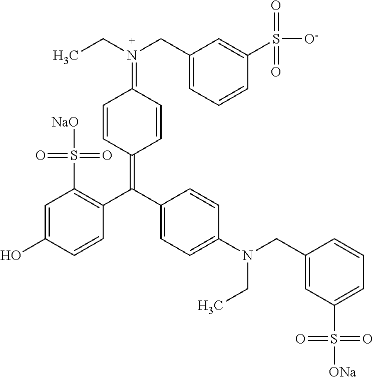

Another illustrative catalyst may be C.I. Acid Blue 90, having a CAS registry number of 6104-58-1 and the following chemical structure:

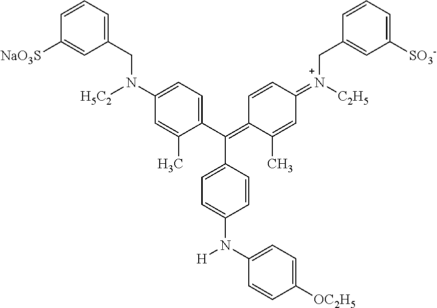

Another illustrative catalyst may be C.I. Food Blue 2, having a CAS registry number of 2650-18-2 and the following chemical structure:

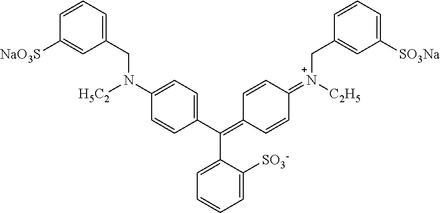

Another illustrative catalyst may be Acid Green 5, having a CAS registry number of 5141-20-8 and the following chemical structure:

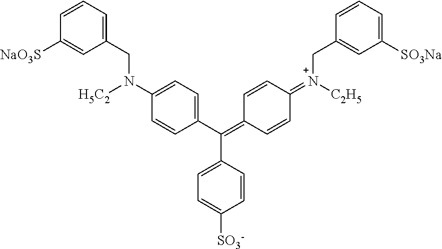

Another illustrative catalyst may be Acid Violet 49, having a CAS registry number of 1694 Sep. 3 and the following chemical structure:

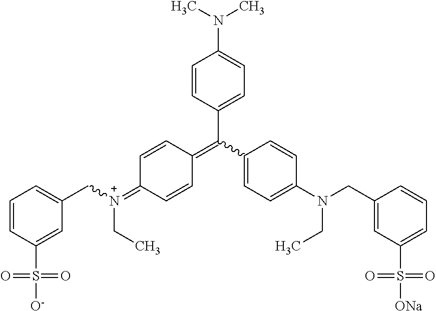

Another illustrative catalyst may be Acid Green 9, having a CAS registry number of 4857-81-2 and the following chemical structure:

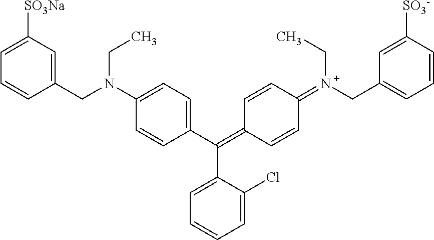

Other Acid Green dyes having an aryl sulfonic acid group may be used as well. Another illustrative catalyst may be Acid Blue 9, having a CAS registry number of 3844-45-9 and the following chemical structure:

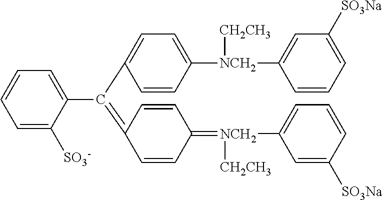

Another illustrative catalyst may be Acid Violet 17, having a CAS registry number of 4129-84-4 and the following chemical structure:

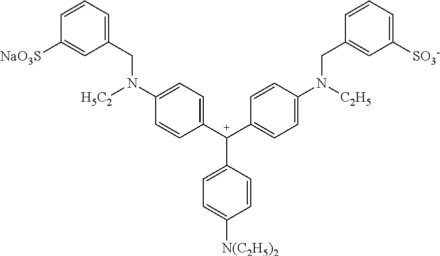

Another illustrative catalyst may be Eriochrome Black T, having a CAS registry number of 1787-61-7 and the following chemical structure:

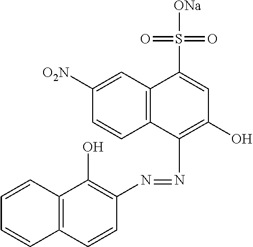

Another illustrative catalyst may be Ponceau S, having a CAS registry number of 6226-79-5 and the following chemical structure:

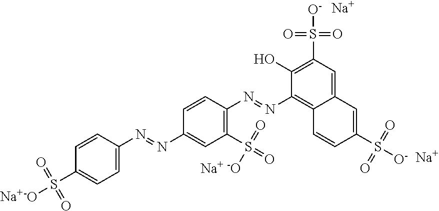

Another illustrative catalyst may be Direct Red 80, having a CAS registry number of 2610 Oct. 8 and the following chemical structure:

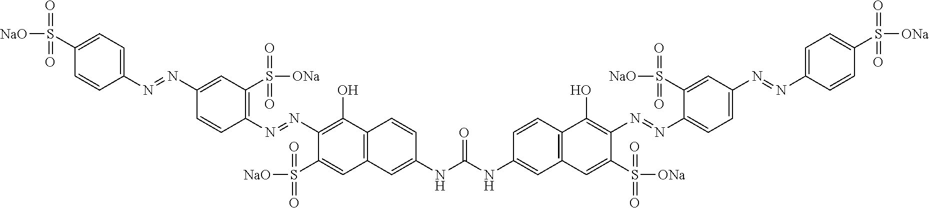

The above catalysts are just some examples of illustrative catalysts that may be used with a base material to form a sterilizing gas abatement member 20. When using dyes as the catalyst, the dye molecules may be in salt form (e.g., sodium salt) as the salt form has greater water solubility. The dye may need to be acidified to convert the sulfonate groups to the more catalytically active sulfonic acid form. This acidification may be performed using an acid with a pKa lower than the dye. Some suitable acids may include, but are not limited to, para-toluenesulfonic acid, fluorosulfonic acid, methanesulfonic acid, trifluoromethanesulfonic acid, or the like. It is contemplated that the dye may be acidified prior to adding the dye (e.g., catalyst) to the base material/acetic acid/water solution described above. In some embodiments, for example, when the base material will not significantly degrade under acidic conditions, acidification may also be done during the mixing of the base material/acetic acid/water solution with the dye to produce the foam. An illustrative acidification reaction of Acid Green 5 is shown as follows:

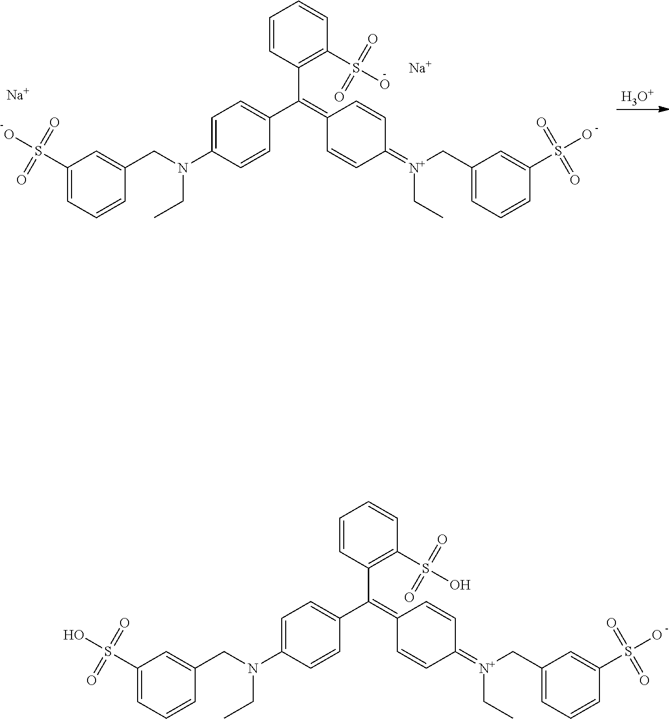

In some cases, when acidified dyes are used as the catalyst, the dyes may form ionic bonds with the base material. However, it is contemplated that there may be less chain entanglement when dyes are used compared to a polymer, such as, but not limited to, PSSA. For example, dyes may have mid-size to macro-size molecules which may result in less chain entanglement. Sterilizing gas abatement members 20 which utilize dyes as the catalyst may be softer, or have a lower durometer, than sterilizing gas abatement members 20 which utilize polymers.

In some examples, the colors emitted by the dyes may tint or color the sterilizing gas abatement member 20. Some dyes may be pH sensitive. Thus, if the catalyst starts to foul (e.g., if somehow the sulfonic acid group is lost) the pH may change along with the color of the sterilizing gas abatement member 20. A color change may provide a visual indication that the sterilizing gas abatement member 20 is losing or has lost the ability to abate ethylene oxide. In some cases, the sterilizing gas abatement member 20 may be provided with a color scale that may be compared to the sterilizing gas abatement member 20 to determine when the sterilizing gas abatement member 20 has lost effectiveness.

In some embodiments, the catalyst and the base material may be formed as separate layers to form the gas abatement member 20. For example, a first layer may be formed from the base material. Illustrative base materials may include, but are not limited to, chitosan, cellulose, cellulose derivatives, other polysaccharides, glycosaminoglycans (e.g., hyaluronic acid or heparin), alginates, or the like. In some cases, the base material may be biodegradable. The base material may have a porosity (e.g., percentage of void space relative to its total volume) in the range of about 10-90%. However, the porosity may be less than 10% or greater than 90%, as desired. A second layer may be formed from a catalyst having mineral acid functional groups which, in some cases, may be, but are not limited to, sulfonic acid groups, sulfuric acid groups, or phosphoric acid groups. Some illustrative catalysts may include, but are not limited to, polystyrene sulfonic acid (PSSA), dyes with sulfonic acid end groups, or the like.

The catalyst layer may be applied to one or more surfaces of the base material by various coating methods, such as, but not limited to, spray coating, dip coating, brush coating, roll coating, or other suitable application techniques. In one illustrative example, spray coating may include atomizing and depositing a solution containing the catalyst on one or more surfaces of the base material. It is contemplated that the catalyst solution may include the catalyst dissolved or suspended in a solvent, such as, but not limited to water, methanol, ethanol, or other appropriate solvents. The concentration of the catalyst in the coating solution may range from about 0.5% to about 20% by weight, although concentrations outside this range are contemplated.

In another illustrative example, to dip coat the base material, the base material may be immersed in a catalyst solution and then withdrawn at a controlled rate to achieve a desired coating thickness. The coated base material may then be dried using methods such as air drying, oven drying at temperatures ranging from about 20° C. to about 100° C., or vacuum drying. Multiple coating and drying cycles may be performed to achieve a desired catalyst loading.

The sterilizing gas abatement substance 20 may be positioned in close proximity to the sterilized primary package 14 to scavenge ethylene oxide gas as it diffuses from the primary package 14. However, this is not required. In the illustrated embodiments of FIG. 1, the sterilizing gas abatement substance 20 may surround the primary package 14 or the primary package 14 may be disposed within the sterilizing gas abatement substance 20. The porous structure of the sterilizing gas abatement member 20 may increase the surface area or provide a large surface area for the ethylene oxide gas to diffuse into and/or through. Additionally, a porous material may allow for a minimal pressure difference across the sterilizing gas abatement substance 20 to allow for diffusion of the ethylene oxide gas into the sterilizing gas abatement substance 20. The high surface area may maximize the potential contact between the reactive sulfonic acid groups and the ethylene oxide gas. This may make the abatement process more efficient. However, it is not required that the sterilizing gas abatement member 20 be porous.

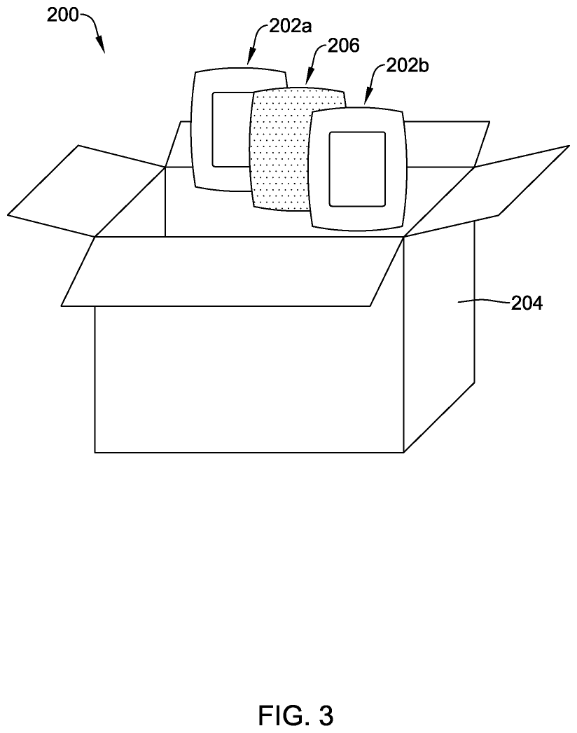

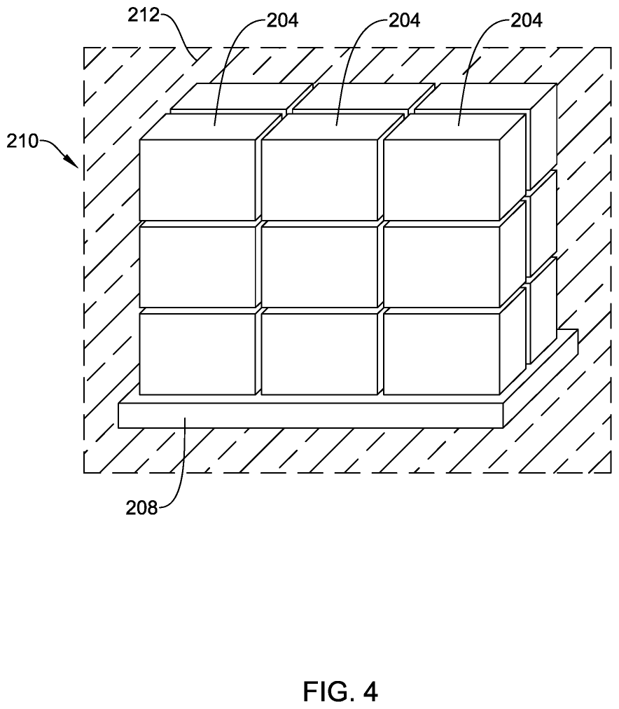

FIG. 3 illustrates a system 200 including a plurality of medical device packages 202a, 202b. The medical device packages 202a, 202b may be similar in form and function to the medical device package 12 described herein. If so provided, an outer package/pouch may or may not be formed from a sterilizing gas abatement substance or member 20, as desired. The medical device packages 202a, 202b may be disposed within a container 204 such as box. The container 204 may include one or more medical device packages 202a, 202b. In some instances, multiple containers 204 may be stacked together onto a pallet 208 as shown in FIG. 4. It can be appreciated that assemblies such as a plurality of medical device packages (e.g., medical device packages 202a, 202b), a plurality of medical device packages within a box/container 204, a plurality of boxes/containers 204 disposed on a pallet 208 could have the ability to release a relatively large amount of ethylene oxide gas after sterilization. Disposing a sterilizing gas abatement member 206 adjacent to the medical device packages 202a, 202b may significantly reduce the level of residual ethylene oxide gas present at or near such assemblies. In some cases, the sterilizing gas abatement member 206 may be provided as a separate component from the medical device packages 202a, 202b. For example, one or more sterilizing gas abatement members 206 may be provided as one or more sheets, blankets, foams, pellets, or other structures within the container 204 as a separate component from the medical device packages 202a, 202b. It is contemplated that the sterilizing gas abatement member 206 may be provided in addition to or in place of a sterilizing gas abatement member 20 surrounding the primary packaging.

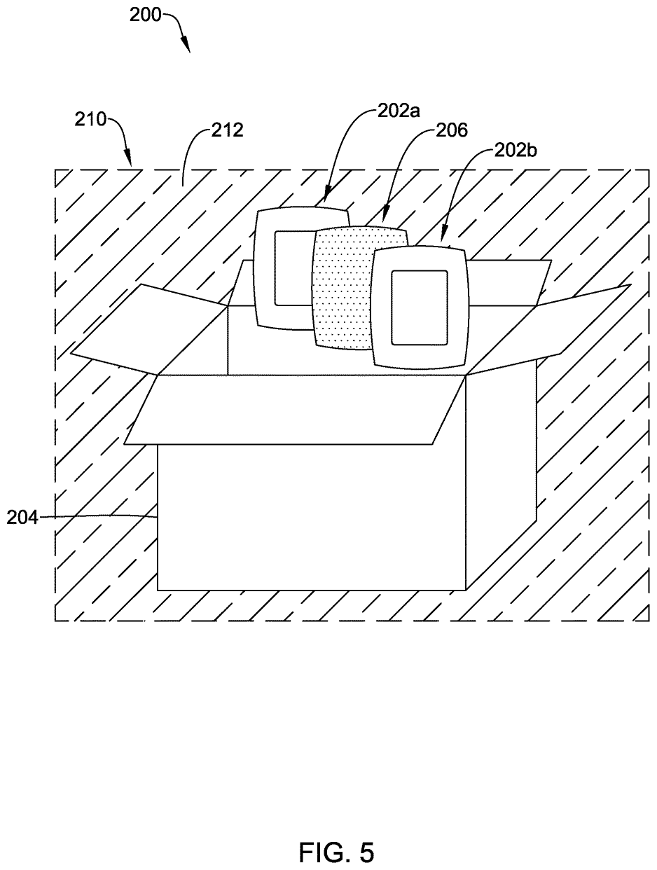

While the sterilizing gas abatement member 20 surrounding the primary packaging and/or the sterilizing gas abatement member 206 disposed within the container 204 may be sufficient to abate residual ethylene oxide gas, other abatement structures are contemplated. For example, FIG. 5 illustrates the system 200 of FIG. 3 including a plurality of medical device packages 202a, 202b disposed within the container 204 along with a sterilizing gas abatement member 206. In this example, a sterilizing gas abatement member 210 may be disposed along, near, or in proximity to the container 204. The sterilizing gas abatement member 210 may take the form of a sterilizing gas abatement blanket having or being formed from a sterilizing gas abatement substance 212. Other form factors are contemplated for the sterilizing gas abatement member 210 includes wraps, boxes, containers, etc. with the sterilizing gas abatement substance 212 incorporated therein. The sterilizing gas abatement member 210 may be provided in place of one or both of the sterilizing gas abatement member 20 and the sterilizing gas abatement member 206 or in addition to one or both of the sterilizing gas abatement member 20 and the sterilizing gas abatement member 206. In some cases, the sterilizing gas abatement member 210 may be a liner placed in the head space of, for example, trucks, shipping containers, or the like, to abate ethylene oxide during transit. Said differently, the sterilizing gas abatement member 210 need not surround the primary packaging or containers 204.

In some instances, the sterilizing gas abatement substance 212 may comprise a porous material that can be incorporated into or entirely form the sterilizing gas abatement member 210. With the sterilizing gas abatement substance 212 woven/incorporated into or otherwise forming the sterilizing gas abatement member 210, the sterilizing gas abatement member 210 can be disposed on, about, form-fitted to, positioned near, etc. various assemblies of medical device packages. For example, the sterilizing gas abatement member 210 can be disposed about, around, surround, or the like, a singular medical device package 202a, 202b. In some of these and in other instances, the sterilizing gas abatement member 210 can be disposed about, around, surround, or the like, a box or container 204 having a plurality of medical device packages 202a, 202b disposed therein (e.g., as shown in FIG. 5). In some instances, multiple containers 204 may be stacked together onto a pallet 208, as shown in FIG. 4. In such instances, the sterilizing gas abatement member 210 may be disposed along, onto, near, or over the pallet 208 and containers 204 to abate ethylene oxide gas. In some cases, the sterilizing gas abatement member 210 may be placed in the head space of, for example, trucks, shipping container, or the like, to abate ethylene oxide during transit.

The sterilizing gas abatement member 210 can suitably scavenge ethylene oxide gas that may diffuse from the medical device packages (e.g., medical device packages 202a, 202b). The sterilizing gas abatement member 210 may have a suitable surface area to cover the medical device packages (e.g., medical device packages 202a, 202b), boxes or containers 204 (e.g., having a plurality of medical device packages 202a, 202b disposed therein), multiple boxes or containers 204 stacked together onto a pallet 208, etc. In addition, the porous nature of the sterilizing gas abatement member 210 (e.g., when such a form factor is utilized) may expand/increase the surface area onto which ethylene oxide can interact with the fabric and, in particular, the sterilizing gas abatement substance 212.

Each of the sterilizing gas abatement members 20, 206 or sterilizing gas abatement member 210 may be formed from the same material or different materials. Each of the sterilizing gas abatement members 20, 206 or sterilizing gas abatement member 210 may include a base material (e.g., chitosan, cellulose, cellulose derivative, or the like) that has a catalyst having sulfonic acid groups that can react with ethylene oxide gas as described herein. Further, the sterilizing gas abatement members 20, 206 or sterilizing gas abatement member 210 may be disposed of prior to use of the packaged medical device.

### Experimental Results

In one illustrative example, three different samples of gas abatement members were manufactured. In a first example, a gas abatement member was formed using cellulose wrapping as the base material and PSSA as the catalyst. In the first example, two (2) grams to cellulose wrapping was sprayed with a 5% PSSA/water solution to ensure the cellulose wrapping was fully wet. The cellulose wrapping was then dried at room temperature. In a second example, a gas abatement member was formed using 50:50 high molecular weight (HMw) chitosan and PSSA. In a third example, a gas abatement member was formed using 50:50 low molecular weight (LMw) chitosan and PSSA. The second and third samples were formed using the procedure described herein. For example, HMw chitosan and LMw chitosan were each mixed with separate acetic acid and water mixtures using an air mixer. PSSA was then added to each of the HMw chitosan/acetic acid/water solution and LMw chitosan/acetic acid/water solution to form two separate mixtures. PSSA was added to each mixture in a one-to-one molar ratio to the chitosan. The resulting mixtures were poured into trays.

The trays were then placed in a freezer having a temperature at or below the freezing point of water (e.g., 32° F. or 0° C.). Once the mixture was completely frozen, the trays were transferred to a freeze dryer for lyophilization. The freeze-drying process continued until the materials appeared dry. Dryness was confirmed when the material was easily removed from the mold.

Seven 20-mL vials were prepared for analysis. A first vial was injected with 5 microliter (μL) ethylene oxide standard with a concentration of 50 milligrams (mg) of ethylene oxide dissolved in methanol in liquid phase. The concentration of the ethylene oxide was equivalent to 12.5 parts per million (ppm) in the 20-mL vial. This vial was heated for one minute at 40° C. to convert the ethylene oxide dissolved in methanol to the gas phase before being injected into gas chromatography. The first vial without ethylene oxide abatement members was used to set a control baseline for a starting concentration of ethylene oxide in each vial to determine a percentage of ethylene oxide remaining over a period of time.

The second vial contained a 50 mg piece of freeze-dried LMw chitosan. The third vial contained 50 mg of HMw chitosan powder. The fourth vial contained 100 mg cellulose wrapping. The fifth vial contained a 40 mg ethylene oxide abatement member formed with cellulose wrapping and PSSA. The sixth vial contained a 54 mg ethylene oxide abatement member formed with HMw chitosan and PSSA. The seventh vial contained a 50 mg ethylene oxide abatement member formed with LMw chitosan and PSSA. Just prior insertion into the gas chromatograph, a volume of the ethylene oxide dissolved in methanol was injected into the second vial. The volume of the ethylene oxide dissolved in methanol was the same as the first vial. Again, the second vial was heated for one minute at 40° C. to convert the ethylene oxide dissolved in methanol to the gas phase. Ethylene oxide concentrations were obtained at intervals of approximately seven minutes. The measured concentrations were compared to the ethylene oxide standard generated with the first vial to determine a percentage of ethylene oxide remaining in the second vial. This was repeated for each of the third, fourth, fifth, sixth, and seventh vials.

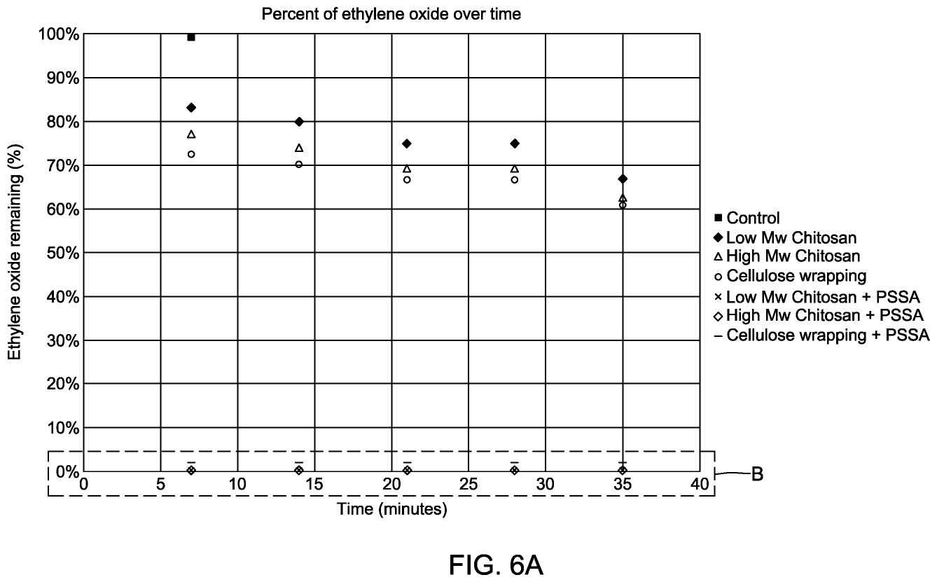

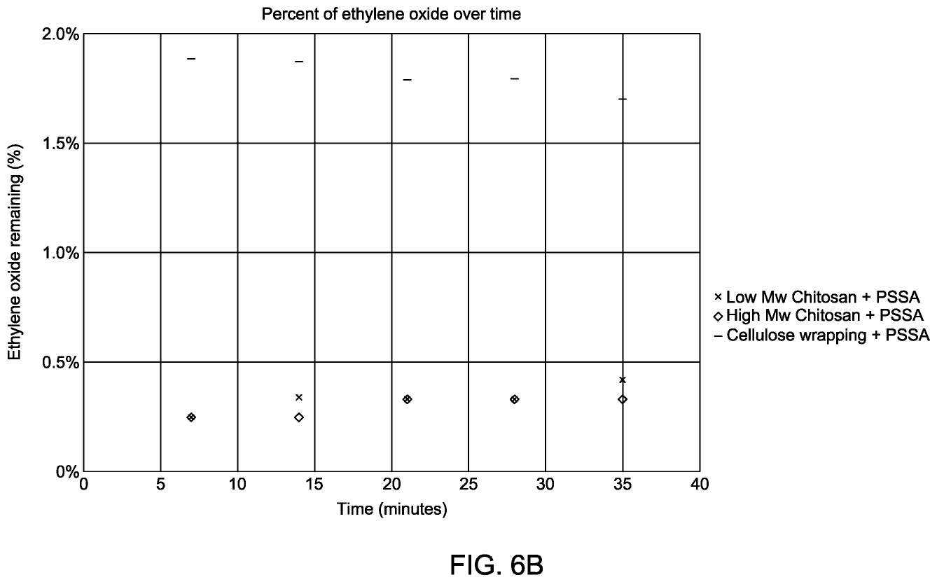

FIG. 6A is a graphical representation of experimental data providing relative ethylene oxide concentrations over a period of time. FIG. 6B is an enlarged region of the graph of FIG. 6A taken at detail B. Test results demonstrated that the ethylene oxide abatement member materials containing PSSA can reduce ethylene oxide levels by approximately 98-99%, or more, within a short time period (e.g., in less than 10 minutes in some cases), whereas control materials without the catalyst component showed minimal reduction in ethylene oxide levels over the time period. Table 1 shows the percent of ethylene oxide over time as measured at the gas chromatograph.

TABLE 1
Percent of ethylene oxide over time

| | | added EO, | Ethylene Oxide remaining after | | | | |
|---|---|---|---|---|---|---|---|
| Materials | Mass, mg | ppm* | 7 mins | 14 mins | 21 mins | 28 mins | 35 mins |
| EO standard | — | 12.5 | 99.2% | — | — | — | — |
| Low Mw Chitosan | 50 | 12.5 | 83.2% | 79.9% | 74.9% | 70.2% | 66.8% |
| High Mw Chitosan | 50 | 12.5 | 77.0% | 74.0% | 69.2% | 66.0% | 62.6% |
| Cellulose wrapping | 100 | 12.5 | 72.5% | 70.2% | 66.6% | 64.6% | 60.9% |
| Low Mw Chitosan + | 50 | 12.5 | 0.2% | 0.3% | 0.3% | 0.4% | 0.4% |
| PSSA | | | | | | | |
| High Mw Chitosan + | 54 | 12.5 | 0.2% | 0.2% | 0.3% | 0.3% | 0.3% |
| PSSA | | | | | | | |
| Cellulose wrapping + | 40 | 12.5 | 1.9% | 1.9% | 1.8% | 1.7% | 1.7% |
| PSSA | | | | | | | |

*5 μL EO standard (certified concentration 50 mg/mL in methanol) was added to 20 mL vials.

In another example, six ethylene oxide abatement members were manufactured using HMw chitosan as the base material and PSSA as the catalyst. The ethylene oxide abatement members were formed by mixing chitosan with an acetic acid and water mixture using an air mixer. PSSA was then added to the chitosan/acetic acid/water solution to form a mixture. PSSA was added to the mixture in a one-to-one molar ratio to the chitosan. The resulting mixture was poured into trays to form the desired shape.

The trays were then placed in a freezer having a temperature at or below the freezing point of water (e.g., 32° F. or 0° C.). Once the mixture was completely frozen, the trays were transferred to a freeze dryer for lyophilization. The freeze-drying process continued until the materials appeared dry. Dryness was confirmed when the material was easily removed from the mold.

The six ethylene oxide abatement members were sized and shaped to be positioned and secured along the outer surface of each side of a sealed cardboard box to surround or wrap the cardboard box with the ethylene oxide abatement members. The edges of the box were tightly sealed using paraffin tape to minimize leakage. The cardboard box with the ethylene oxide abatement members was placed in a warehouse with pallets of devices that had been previously sterilized with ethylene oxide and were off-gassing. The ambient ethylene oxide concentrations in the warehouse exceeded 100 ppb. A portable high-resolution gas FTIR (Fourier Transform Infrared Spectroscopy) with a limit of quantification of 1 ppb was used to take measurements of ethylene oxide concentrations outside of the cardboard box. The sample line for the gas FTIR was then placed inside of the cardboard box surrounded by the ethylene oxide abatement members. Due to the small internal volume of the box (<4 liters), the sampling system pulled ambient air through the porous foam matrix at a rate of 3-4 liters per minute. Over the course of the experiment, this setup filtered hundreds of liters of ethylene oxide-laden air through the foam.

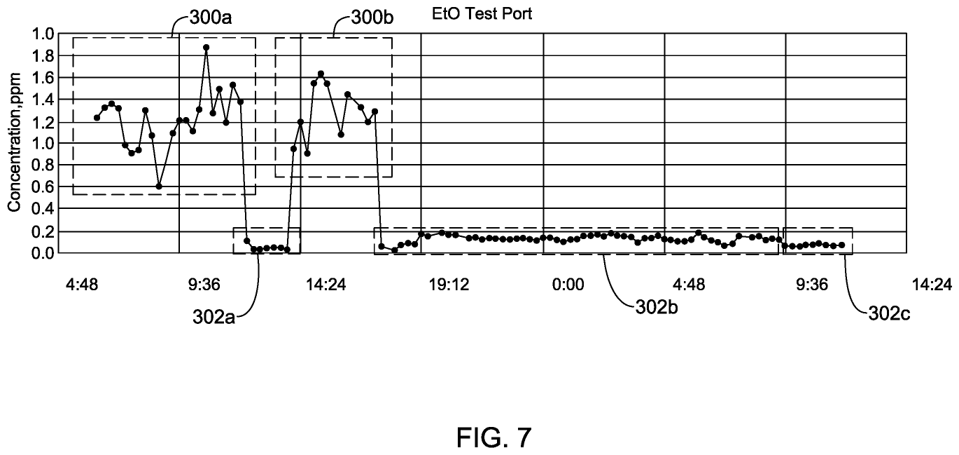

FIG. 7 is a graphical representation of experimental data providing relative ethylene oxide concentrations of the ambient atmosphere and inside the wrapped cardboard box over a period of time. To begin, the gas FTIR was positioned exterior to the wrapped cardboard box. As can be seen in FIG. 7, the concentration of ethylene oxide in the ambient atmosphere (e.g., exterior to the wrapped cardboard box) in the warehouse was in the range of about 0.6 to about 1.8 ppm with an average concentration of around 1.3 ppm, as shown at boxed region 300a. Once a baseline ethylene oxide concentration was obtained, the gas FTIR was inserted into an interior of the wrapped cardboard box. When the sample line was placed within an interior of the wrapped cardboard box, the measured ethylene oxide concentration dropped to around 0.2 ppm or less, as shown at boxed region 302a. The sample line was then moved back to the ambient atmosphere where the ethylene oxide concentration was again measured in the 0.8 to about 1.7 ppm range, as shown at boxed region 300b. The sample line was reinserted into the interior of the wrapped box where the ethylene oxide concentration was again measured at around 0.2 ppm or less, as shown at boxed regions 302b, 302c. The chitosan-PSSA foam exhibited strong catalytic activity toward EtO, leading to significant surface adsorption. As a result, only trace amounts of EtO were detected inside the box, indicating that the foam effectively captured and reduced the gas before it could penetrate the enclosed space.

Near the end of the data collection, a gap was identified near where the sample line entered the box. This gap appears to have allowed ambient air to enter the interior of the wrapped box without contacting any of the ethylene oxide abatement members. The gap was sealed with paraffin tape. Once the gap was sealed, the concentration of the ethylene oxide within the interior of the wrapped box dropped even lower as shown at boxed region 302c indicating that the ethylene oxide abatement members were effectively removing ethylene oxide from the atmosphere. Further, as a control, the sample line was placed within an interior of an unwrapped cardboard box (free from any ethylene oxide abatement members). The ethylene oxide concentration within the interior of the unwrapped box was similar to the ambient environment further demonstrating the effectiveness of the ethylene oxide abatement members. Table 2 summarizes the relative decrease in ethylene oxide concentration within the cardboard box surrounded by the abatement members. The results are separated as trial 1, a time before the gap was identified and shown at 302a in FIG. 7, trial 2, a time when ambient air was able to enter the interior of the box via the gap and shown at 302b in FIG. 7, and trial 3, a time after the gap was sealed and shown at 302c in FIG. 7.

TABLE 2
Decrease in ethylene oxide concentrations

| | Outside EtO | Inside EtO | Reduction | |
|---|---|---|---|---|
| | Conc. | Concentration | Efficiency | |
| | (ppm) | (ppm) | (%) | Notes |
| Trial 1 | 1.2 | 0.036 | 97 | N/A |
| Trial 2 | 1.3 | 0.136 | 90 | Small leak identified |
| Trial 3 | 1.3 | 0.059 | 95 | Leak sealed using |
| | | | | Paraffin tape |

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for abatement of sterilizing gas, the system comprising:
a device package having a device disposed therein; and
a sterilizing gas abatement member disposed adjacent to the device package, the sterilizing gas abatement member comprising:
a base material; and
a catalyst comprising sulfonic acid functional groups;
wherein the catalyst is configured to abate a sterilizing gas present adjacent to the device package.

2. The system of claim 1, wherein the base material comprises at least one of chitosan, cellulose, or a cellulose derivative, other polysaccharides, glycosaminoglycans, or other related systems.

3. The system of claim 1, wherein the catalyst comprises polystyrene sulfonic acid (PSSA).

4. The system of claim 1, wherein the sterilizing gas abatement member has a porosity in a range of about 10-90%.

5. The system of claim 4, wherein the sterilizing gas abatement member has a porosity in a range of about 40-80%.

6. The system of claim 1, wherein the sterilizing gas abatement member is configured as a blanket.

7. The system of claim 1, wherein the sterilizing gas abatement member is configured as a liner.

8. The system of claim 1, wherein the sterilizing gas comprises ethylene oxide.

9. The system of claim 1, wherein the device package is disposed within the sterilizing gas abatement member.

10. The system of claim 1, wherein the sterilizing gas abatement member is disposed about the device package.

11. A sterilizing gas abatement system comprising:
a container configured to hold one or more sterilized devices;
a sterilizing gas abatement member positioned within or surrounding the container, the sterilizing gas abatement member comprising:
a porous matrix formed from a base material and a catalyst;
wherein the base material provides structural integrity to the porous matrix;
wherein the catalyst comprises sulfonic acid functional groups; and
wherein the catalyst is configured to convert ethylene oxide gas to ethylene glycol.

12. The sterilizing gas abatement system of claim 11, wherein the porous matrix has a porosity in the range of about 10-90%.

13. The sterilizing gas abatement system of claim 11, wherein the base material comprises at least one of chitosan, cellulose, or a cellulose derivative, other polysaccharides, glycosaminoglycans, or other related systems.

14. The sterilizing gas abatement system of claim 11, wherein the catalyst comprises polystyrene sulfonic acid (PSSA).

15. The sterilizing gas abatement system of claim 11, wherein the catalyst is present in a molar ratio of one-to-one or less to the base material.

16. A method of manufacturing a sterilizing gas abatement member, the method comprising:
mixing a base material with an acid and water solution to form a first mixture;
adding a catalyst comprising sulfonic acid functional groups to the first mixture to form a second mixture;
pouring the second mixture into a mold; and
removing water from the second mixture to form a porous matrix.

17. The method of claim 16, wherein removing water comprises freeze-drying the second mixture.

18. The method of claim 16, wherein removing water comprises at least one of: spray drying, oven drying, vacuum drying, fiber spinning, or using supercritical CO2.

19. The method of claim 16, wherein the catalyst is added in a molar ratio of one-to-one or less to the base material.

20. The method of claim 16, wherein the acid comprises acetic acid, lactic acid, or hydrochloric acid.
